(19) United States
(12) Patent Application Publication
(10) Pub. No.: US 2005/0136395 A1
(43) Pub. Date: Jun. 23, 2005

(54) Methods for genetic analysis of SARS virus

(75) Inventors: Michael P. Mittmann, Palo Alto, CA (US); Eric B. Schell, Mountain View, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC, Santa Clara, CA (US)

(21) Appl. No.: 10/843,527

(22) Filed: May 10, 2004

Related U.S. Application Data
(60) Provisional application No. 60/469,545, filed on May 8, 2003.

(51) Int. Cl. C12Q 1/70; C12Q 1/68; C12P 19/34

(52) U.S. Cl. 435/5; 435/6; 435/91.2

(57) ABSTRACT

The invention provides arrays and probes for resequencing a SARS virus using an array of probes that are complementary to a SARS reference sequence and to each possible single nucleotide substitution of the reference sequence. Methods of identifying mutations in viral sequences and methods of characterizing viral isolates are also provided. The invention also provides high throughput methods to monitor epidemics and pandemics caused by pathogens such as viruses.

### RELATED APPLICTIONS

The present application claims priority to U.S. Provisional Application No. 60/469,545, filed May 8, 2003, the disclosure of which is incorporated herein by reference in its entirety.

### FIELD OF THE INVENTION

Pools of nucleic acid sequences and arrays of nucleic acid sequences that are useful for detecting sequence variation in the SARS virus are provided. High throughput methods for using sequence variation detection for analysis and monitoring of viruses and viral outbreaks are provided. The invention relates to diverse fields, including viral genetics, epidemiology, medicine, and medical diagnostics.

### REFERENCE TO SEQUENCE LISTING

The Sequence Listing submitted on compact disk is hereby incorporated by reference. The file on the disk is named 3602.1seqlist.txt the file is 32.8 MB and the date of creation is May 10, 2004.

### BACKGROUND

Severe acute respiratory syndrome (SARS) captured the attention of the world in the Spring of 2003. The syndrome arose in China in late 2002 and was spread around the world by travelers. Nearly 800 people were killed and 8,000 infected during the initial outbreak. This previously unknown disease has had its most severe impact in Hong Kong and China, but has also been identified in patients in Canada, the United States, Europe and other Asian countries. The rapid global spread of the disease highlights the increasing risk for pandemics created by increasing globalization and travel. An individual infected with a disease can easily travel to any major city in a matter of hours. Monitoring disease outbreaks of both old diseases such as influenza and new diseases such as SARS will continue to be important to world health and stability. Single nucleotide polymorphism (SNP) has been used extensively for genetic analysis.

Fast and reliable hybridization-based polymorphism detection assays have been developed. (See Wang, et al., Large-Scale Identification, Mapping, and Genotyping of Single-Nucleotide Polymorphism's in the Human Genome, Science 280:1077-1082, 1998; Gingeras, et al., Simultaneous Genotyping and Species Identification Using Hybridization Pattern Recognition Analysis of Generic Mycobacterium DNA Arrays, Genome Research 8:435-448, 1998; Halushka, et al., Patterns of Single-Nucleotide Polymorphisms in Candidate Genes for Blood-Pressure Homeostasis, Nature Genetics 22:239-247, 1999; Cutler, et al., High throughput variation detection and genotyping using microarrays. Genome Research 11(11): 1913-25, 2001, (Cutler et al., 2001) all incorporated herein by reference in their entireties.

### SUMMARY OF THE INVENTION

In one embodiment a microarray for resequencing different isolates of the coronavirus-like virus that causes severe acute respiratory syndrome (SARS) is disclosed. The array may comprise 1 or more probes corresponding to SEQ ID NOS. 1-238,192. In one embodiment the array comprises probes corresponding to each of the sequences in SEQ ID NOS. 1-238,192 and may in addition comprise a collection of control probes, for example, Tag-IQ-EX and 60 mer as disclosed in U.S. patent application Ser. No. 10/619,739.

The probes for the array were selected using the publicly available SARS sequences published by researchers at the British Columbia Cancer Center, the US Centers for Disease Control sequence, and sequences published by scientists from Asia.

Probes are included for the entire 29,700 nucleotide sequence of the SARS virus. The array is designed for rapid resequencing of the virus. The array may be used to identify mutations in the virus from different isolates. Resequencing may be used to categorize viral isolates into subtypes, and also, to compare the sequence to specific clinical data. In some embodiments sequence may be combined with additional data, such as clinical data, to determine why one strain is more dangerous than another. The sequence obtained may also be used to track the virus' evolution over time in different populations and different areas.

The invention provides an array of oligonucleotide probes immobilized on a solid support for analysis of a target sequence from a coronavirus. The array comprises at least four sets of oligonucleotide probes 9 to 35 nucleotides in length. In a preferred embodiment probes are 20 nucleotides in length. In another preferred embodiment probes are 25 nucleotides in length. A first probe set has a probe corresponding to each nucleotide in a reference sequence from a SARS virus. A probe is related to its corresponding nucleotide by being exactly complementary to a subsequence of the reference sequence that includes the corresponding nucleotide. Thus, each probe has a position, designated an interrogation position, that is occupied by a complementary nucleotide to the corresponding nucleotide. The three additional probe sets each have a corresponding probe for each probe in the first probe set. Thus, for each nucleotide in the reference sequence, there are four corresponding probes, one from each of the probe sets. The three corresponding probes in the three additional probe sets are identical to the corresponding probe from the first probe or a subsequence thereof that includes the interrogation position, except that the interrogation position is occupied by a different nucleotide in each of the four corresponding probes. Both strands of the sequence may be tiled on an array in this manner to detect polymorphism on either or both strands.

In another aspect, the invention provides methods for comparing a target nucleic acid from a SARS virus with a reference sequence from a second SARS virus having a predetermined sequence of nucleotides. The target nucleic acid is hybridized to an array of oligonucleotide probes as described above. The relative specific binding of the probes in the array to the target is determined to indicate whether the target sequence is the same or different from the reference sequence.

In some applications, the target sequence has a substituted nucleotide relative to the reference sequence in at least one undetermined position, and the relative specific binding of the probes indicates the location of the position and the nucleotide occupying the position in the target sequence. In some applications the target sequence has a substituted nucleotide relative to the reference sequence in at least one position, the substitution conferring drug resistance to the SARS virus, and the relative specific binding of the probes reveals the substitution.

In some embodiments methods of monitoring viral isolates from different individuals are disclosed. Many of the methods may have at least one step that is performed in a high throughput assay that may involve one or more robots. Many of the methods involve resequencing a viral isolate on a SARS resequencing array and comparing the sequence of the isolate to one or more other sequences. In some embodiments the frequency of a particular mutation is determined. In some embodiments a particular mutation or mutations may be associated with a phenotype, for example, a super spreader phenotype.

### DETAILED DESCRIPTION OF THE INVENTION

(A) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,153,743, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com. Example arrays are shown on the website at affymetrix.com. Arrays are disclosed in U.S. Pat. Nos. 6,610,482.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H.A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), 10/065,856, 10/065,868, 10/328,818, 10/328,872, 10/423,403, and 60/482,389.

B. Definitions

The phrase “massively parallel screening” refers to the simultaneous screening of from about 100, 1000, 10,000 or 100,000 to 1000, 10,000, 100,000, 1,000,000 or 3,000,000 or more different nucleic acid hybridizations.

As used herein a “probe” is defined as a nucleic acid capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, a linkage other than a phosphodiester bond may join the bases in probes, so long as it does not interfere with hybridization. Thus, probes may be peptide nucleic acids in which the constituent bases are joined by peptide bonds rather than phosphodiester linkages.

The term “match,” “perfect match,” “perfect match probe” or “perfect match control” refers to a nucleic acid that has a sequence that is designed to be perfectly complementary to a particular target sequence. The nucleic acid is typically perfectly complementary to a portion (subsequence) of the target sequence. A perfect match (PM) probe can be a “test probe”, a “normalization control” probe, an expression level control probe and the like. A perfect match control or perfect match is, however, distinguished from a “mismatch” or “mismatch probe.”

The term “mismatch,” “mismatch control” or “mismatch probe” refers to a nucleic acid whose sequence is deliberately designed not to be perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions. A homo-mismatch substitutes an adenine (A) for a thymine (T) and vice versa and a guanine (G) for a cytosine (C) and vice versa. For example, if the target sequence was: AGGTCCA, a probe designed with a single homo-mismatch at the central, or fourth position, would result in the following sequence: TCCTGGT.

A genetic map is a map that presents the order of specific sequences on a chromosome.

Genetic variation refers to variation in the sequence of the same region between two or more organisms.

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 20 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

A genome is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

The term “chromosome” refers to the heredity-bearing gene carrier of a cell which is derived from chromatin and which comprises DNA and protein components (especially histones). The conventional internationally recognized individual human genome chromosome numbering system is employed herein. The size of an individual chromosome can vary from one type to another within a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ bp while the smallest chromosome, chromosome no. 22, contains about 5.3×10⁷ bp.

A “chromosomal region” is a portion of a chromosome. The actual physical size or extent of any individual chromosomal region can vary greatly. The term “region” is not necessarily definitive of a particular one or more genes because a region need not take into specific account the particular coding segments (exons) of an individual gene.

An “allele” refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”. At each autosomal specific chromosomal location or “locus” an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

Polymorphism refers to the occurrence of two or more genetically determined alternative sequences or alleles in a population. A polymorphic marker or site is the locus at which divergence occurs. Preferred markers have at least two alleles, each occurring at a frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

Single nucleotide polymorphisms (SNPs) are positions at which two alternative bases occur at appreciable frequency (>1%) in a given population. SNPs are the most common type of human genetic variation. A polymorphic site is frequently preceded by and followed by highly conserved sequences (e.g., sequences that vary in less than 1/100 or 1/1000 members of the populations).

A SNP may arise due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

The term “genotyping” refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Those individuals who have two copies of the C allele are homozygous for the C allele, those individuals who have two copies of the A allele are homozygous for the C allele, and those individuals who have one copy of each allele are heterozygous. The array may be designed to distinguish between each of these three possible outcomes. A polymorphic location may have two or more possible alleles and the array may be designed to distinguish between all possible combinations.

As used herein, a “probe” is a molecule for detecting a target molecule. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as the bond does not prevent hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991), each of which is incorporated by reference in its entirety for all purposes.

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.)

A resequencing array is an array of nucleic acid probes with four probes tiled for both the forward and reverse strand for each individual base in a sequence. The central position of each probe varies to incorporate each of the four possible nucleotides, A, C, G or T. See, GeneChip CustomSeq Resequencing Arrays Data Sheet, available from Affymetrix, Inc. part no. 701225 Rev. 2. Arrays are designed based on the sequence to be resequenced. A known sequence is selected and the array is designed using that sequence as a reference sequence.

Assays for amplification of the known sequence are also designed, for example primers for long range PCR may be designed to amplify regions of the sequence. For RNA viruses a first reverse transcriptase step may be used to generate double stranded DNA from the single stranded RNA. A resequencing array for the SARS virus was designed to resequence the approximately 29,000 base sequence published for the SARS virus. The resequencing array may be designed to resequence an entire genome, such as the genome of the SARS virus; one or more regions of a genome, for example, selected regions of a genome such as those coding for a protein or RNA of interest; a conserved region from multiple genomes; or multiple genomes, such as the genome of a first SARS isolate and the genome of a second SARS isolate, or the genome of SARS and the genome of a second coronavirus. Resequencing arrays and methods of genetic analysis using resequencing arrays is described in Cutler, et al, Genome Res. 11(11): 1913-25 (2001) and Warrington, et al., Hum Mutat 19:402-9 (2002) and in US Patent Pub. No. 20030124539 each of which is incorporated herein by reference in its entirety.

A resequencing array has probes to a reference sequence from a SARS virus tiled so that each nucleic acid position in the reference sequence is interrogated by a probe set of at least four perfect match probes. Each of the four probes is a perfect match to a different sequence and the sequences differ at the interrogation position, which is typically the central base of the probe. For example, nucleotide 13 in a 25 nucleotide probe. The first probe of the four probes is perfectly complementary to the reference sequence and each of the remaining three probes is perfectly complementary to a different single base mutation at the interrogation position so that at least one probe of the four probes is perfectly complementary to each of the four possible bases present at the interrogation position.

Arrays may be packaged in such a manner as to allow for diagnostic use or can be an all-inclusive device; e.g., U.S. Pat. Nos. 5,856,174 and 5,922,591 incorporated in their entirety by reference for all purposes. Preferred arrays are commercially available from Affymetrix (Santa Clara, Calif.) under the brand name GeneChip® and are directed to a variety of purposes, including genotyping and gene expression monitoring for a variety of eukaryotic and prokaryotic species. The number of probes on a solid support may be varied by changing the size of the individual features. In some embodiments the feature size is 20 by 25 microns square, in other embodiments features may be, for example, 8 by 8, 5 by 5 or 3 by 3 microns square, resulting in about 2,600,000, 6,600,000 or 18,000,000 individual probe features.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. patent application Ser. No. 08/630,427, filed Apr. 3, 1996.

The term hybridization refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example tow complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature and the concentration of salt in the reaction. Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) or 100 mM MES, 1 M Na, 20 mM EDTA, 0.01% Tween-20 and a temperature of 25-50° C. are suitable for allele-specific probe hybridizations. In a particularly preferred embodiment hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

C. SARS Virus Resequencing Array

Infections diseases spread by the respiratory route are a serious threat to the health of people around the world. Epidemics of such diseases can be devastating to communities, particularly if there are no effective vaccines or drugs. The recently discovered SARS virus is an example of a dangerous disease epidemic. The disease is easily transmitted, particularly as a nosocomial infection, and has proven fatal in a relatively large percentage of cases. Because of the dangerous nature of this disease measures will need to be taken to prevent and control future epidemics. These measures will benefit from a better understanding of the genome of the virus and variations between different isolates. Researchers identified the virus as a relative of known coronaviruses. See, Zhu and Chen J. Infect. Dis. 189: 1676-8, 2004.

Rapid efforts have been undertaken to determine the nature of the SARS virus which has been identified as the etiological agent of the SARS outbreak of 2003. See, Ksiazek et al. N. Engl. J. Med. 348, 1947 (2003), Peiris et al., Lancet 361, 1319 (2003), and Drosten et al., N. Engl. J. Med. (2003). See also Xu et al. N. Engl. J. Med. 350:1366-7, 2004, Heymann and Rodier, Emerg Infect Dis 10:173-5, 2004 and Hughes, Emerg Infect Dis 10:171-2, 2004.

SARS has been identified by hybridization analysis to be a relative of the coronavirus family, having sequence similarity to several coronaviruses. Subsequently, the entire genomes of several isolates of the virus were sequenced. As with many viruses, the currently available sequencing data suggests that the virus mutates rapidly. The genomes currently available show considerable variation, however the two isolates available from the United States vary only in 8 nucleotides. Variations occur in the enzymes that replicate the virus and in proteins that sit on the outer surface of the viral particle. As with many viruses, mutation allows the virus to defeat the host's defenses and often confer resistance to drugs so it is important to identify mutations and to correlate them with clinical phenotypes. Mutations may also be responsible for differences in pathogenicity and infectivity. The methods presently disclosed may be used to rapidly identify mutations in a sample isolate by comparing that sequence to a reference sequence. The sample isolate is hybridized to an array of probes. The array of probes comprises the entire sequence of the reference genome tiled so that there is a probe to interrogate each position of the sequence for each possible single nucleotide substitution (see U.S. Pat. Nos. 5,837,832 and 5,861,242 which are incorporated herein by reference).

The arrays described herein may be used to determine sequence variation among SARS samples. The arrays and genetic information obtained by using the arrays may also be used to categorize different isolates of the virus into subtypes. Also disclosed are methods of comparing patient outcome with the pathogen subtypes, allowing for better understanding of strains that are the most dangerous and to develop therapies and drugs. Methods are also disclosed for using a resequencing array to determine how a virus, for example, the SARS virus, is evolving over time, during its spread into different geographies and populations.

The methods provide an array of oligonucleotide probes immobilized on a solid support for analysis of a target sequence from a SARS virus. The array comprises oligonucleotide probes 9-35 nucleotides in length. The probes are present in sets of 8 probes that are related. A first referenced probe in the set comprises a sequence corresponding to the sequence of the reference sequence. A second reference probe is the complement of the first probe. This way both strands are analyzed. Three of the remaining 6 probes are identical to the first probe except for a single nucleotide, the interrogation position, which is varied so that each of the possible 4 bases is represented. The remaining 3 probes are identical to the second probe save for variation at the interrogation position to each of the other three possible bases. For example, if the interrogation position has a G in the reference sample there will be a reference probe with a C that is perfectly complementary to the reference sequence, a non-reference probe with an A, a non-reference probe with a G and a non-reference probe with a T at that position, the latter three probes being complementary to mutation at that position to T, C and A respectively. If the interrogation position is mutated hybridization will occur at one of the non-reference probes.

Monitoring viruses, both newly emerging viruses and well established viruses such as influenza is an important measure taken to control the spread an impact of viruses. In some embodiments arrays such as those disclosed could be used for rapid analysis of many viral samples to identify which isolate or isolates are present in an individual and to identify sequence variation between isolates. In some embodiments a single array may be used to resequence or detect known variations in a plurality of different viruses. For example, an array may have probes to one or more SARS strains and one or more influenza strains and subtypes. Arrays may, for example, include probes to detect and distinguish between strains and subtypes of avian influenza, “bird flu”, which can be highly pathogenic, for example influenza A viruses of subtypes H5 and H7. Avian influenza has been known to infect humans. Arrays may be designed to identify variation or detect known variants in the virus that causes foot and mouth disease in cloven-hoofed animals.

Resequencing arrays may also be used in studies to determine the origin of a virus. Viruses that impact humans often originated in other organisms, such as apes or chickens, and make the jump to humans as a result of close human contact with an infected organism. Tracing a virus back to its origins is a difficult task that would require isolation of virus from a variety of potential sources, sequencing of the virus from these sources and comparison of the sequences to look for relatedness. The resequencing array presently disclosed may be used to simplify this process. Virus from many sources could be rapidly resequenced to identify differences using an array as disclosed.

Vaccination has proven to be an effective means of controlling the outbreak of some diseases. The presently disclosed methods may be used as part of a method to develop a vaccine or to determine the effectiveness of a vaccine. One of the ways that viruses evade the immune response is to mutate to a form that is unrecognizable to the antibodies of the response. Mutations in the proteins that make the outer surface or coat of the virus, for example, can change the antigenic response allowing the virus to escape recognition. Resequencing many isolates of a virus may provide information about mutations that confer immune response resistance and provide researchers with information about what regions of the virus are more stable and therefore better antigenic targets.

In one embodiment the methods are used to track transmission of disease from patient to patient or from location to location. Isolates from individuals may be compared to identify those isolates that are similar to determine the likely source of infection. The methods may be used to trace an outbreak to a particular source of infection. For example, a SARS outbreak in 2004 that resulted in nine cases was traced to a medical student who caught SARS while studying SARS in a laboratory. There have been at least three outbreaks of SARS that seem to have originated from laboratory research on the virus. See, Normile, Science 304:659-661, 2004. In one aspect a SARS resequencing array may be used to monitor laboratory personnel or healthcare workers for exposure on a regular basis, daily or weekly, for example, in order to identify infection early. A resequencing analysis of SARS isolates from individuals who were infected from the same source should show a high degree of similarity between the isolates at the sequence level.

In some embodiments the methods may be used to identify and characterize “super spreader” strains, also known as “super transmitter”. See, Riley et al. Science 300:1884-5, 2003. Viral nucleic acid may be isolated from individuals suspected of carrying a more virulent strain of the virus and a resequencing array may be used to identify polymorphisms characteristic of the isolate.

In some embodiments an array may have probes for resequencing one or more viruses or pathogens and probes for genotyping one or more human polymorphisms. During outbreaks of disease such as the SARS outbreak some individuals have an increased likelihood of contracting the disease relative to the general population and some individuals experience symptoms that are more or less severe that expected. Genetic variation likely accounts for variation in the susceptibility to disease and the severity of symptoms. An array that genotypes human polymorphisms that are associated with this variation would allow diagnosis of infection and prediction of outcome simultaneously. For example, if individuals that carry allele A of SNP 1 are more likely to suffer a relapse during a SARS infection than individuals who carry allele B of SNP 1 a single array may be used to determine if the individual has the infection and if the individual is at risk for relapse, suggesting a different course of treatment. Similarly the array may contain probes to genotype one or more SNPs that predict response of an individual to a particular drug treatment regime.

In another embodiment reference sequences characteristic of other pathogens may be included on an array. The symptoms of SARS are common to several other diseases and patients who are suspected of having a SARS infection may be infected with a different pathogen, such as influenza or Mycobacterium tuberculosis. See, for example, CDC, MMWR Morb Mortal Wkly Rep., 53:321-2, 2004.

In some embodiments the methods disclosed eliminate the need to culture the virus outside of the host prior to sequencing. Mutations can accumulate while the virus is being cultured for sequencing. These mutations may be adaptations to laboratory culture and may not have been present in the virus isolated from the patient. Direct analysis of the virus without laboratory cell culture may be performed using the methods presently disclosed. Viral nucleic acid may be isolated from the host, amplified and analyzed on a resequencing array without the need for cell culture.

Epidemics and newly-emerging infections such as SARS are becoming an increasing threat to the health of people around the world and affecting international travel and trade. Factors such as, globalization, the growth of mega cities and increases in international travel amplify the potential for rapid spread of infections. The methods presently disclosed are particularly useful for early, rapid and extensive monitoring of sequence variation occurring in viral isolates. In many embodiments high throughput methods for resequencing viruses are contemplated.

A database of viral sequences may be developed. Resequencing analysis in combination with high throughput methods may be used to generate sequence variation information from a large number of viral isolates. The sequence variation information may be used to generate a database of sequence variation information. The sequence variation information may be coupled to additional information, for example, information about the geographic location where the sample was isolated, clinical information about the patient such as duration of illness, effectiveness of treatment, morbidity, mortality, and degree of transmission and biographical information about the patient, for example, age, gender, health, and other socioeconomic facts.

Early sequence monitoring of many isolates in parallel may be used to rapidly identify isolates and mutations. Some isolates of a given virus may have more severe phenotypes than other isolates, for example, higher morbidity or mortality rates and drug resistance. When a new outbreak occurs rapid resequencing of isolates from affected individuals may be used to identify individuals infected with isolates known to have more dangerous phenotypes and steps can be taken to aggressively contain the spread of those isolates. For example, a plurality of individuals may be identified as having symptoms of a disease. Isolates of the virus responsible from the disease may be isolated from each of the affected individuals and resequenced to identify variation. The variation may be compared against a database of variation and phenotypes associated with these variations to identify individuals who have strains of the virus that are known to be, for example, more easily transmitted than other strains. Aggressive steps may be taken to insure that those individuals infected with the more transmittable strain are isolated so that transmission is limited. Resources may also be allocated to identifying people that were likely to have been contacted by individuals with the easily transmitted strain in order to minimize the spread of strains with more severe phenotypes.

Multiple genomes may be tiled on a single array, for example, one array may have an influenza virus genome and a SARS virus genome. Several different species of a single genus of viruses may be tiled on an array, for example, an array may comprise probes to resequence the SARS coronavirus, human coronavirus, bovine coronavirus, and rat coronavirus. Regions of interest from several different viruses may be tiled on an array, for example, an array may be designed to resequence a replicase gene from several different viruses. Regions of a virus may be selected for resequencing such as the ORFs. Regions of a virus that are known to confer drug resistance may be tiled on a resequencing array, see, for example U.S. Pat. No. 5,861,242. Viral isolates from clinical samples may be resequenced to identify mutation and then the mutations may be correlated with phenotypes such as drug resistance to a particular drug, severity of illness, increased risk of mortality, increased risk of transmission, etc. This information may be used to select a treatment for a patient or to predict length of treatment.

Another virus that may be monitored by the methods disclosed is the influenza virus. Influenza is caused by a virus that attacks mainly the upper respiratory tract—the nose, throat and bronchi and rarely also the lungs. The infection usually lasts for about a week. It is characterized by sudden onset of high fever, myalgia, headache and severe malaise, non-productive cough, sore throat, and rhinitis. Most people recover within one to two weeks without requiring any medical treatment. In the very young, the elderly and people suffering from medical conditions such as lung diseases, diabetes, cancer, kidney or heart problems, influenza poses a serious risk. In these people, the infection may lead to severe complications of underlying diseases, pneumonia and death.

Influenza rapidly spreads around the world in seasonal epidemics and imposes a considerable economic burden in the form of hospital and other health care costs and lost productivity. In the United States of America, for example, recent estimates put the cost of influenza epidemics to the economy at US$ 71-167 billion per year (see World Health Organization web site). Annual influenza epidemics are thought to result in between three and five million cases of severe illness and between 250,000 and 500,000 deaths every year around the world. Vaccination is the principal measure for preventing influenza and reducing the impact of epidemics.

Constant genetic changes in influenza viruses mean that the vaccines' virus composition must be adjusted annually to include the most recent circulating influenza A(H3N2), A(H1N1) and influenza B viruses. The World Health Organization constantly monitors the influenza viruses circulating in humans to rapidly identify new strains. The 3 most virulent strains in circulation are used to generate a vaccine each year. In some embodiments a resequencing array designed to resequence one or more isolates of influenza virus is disclosed and methods for high throughput analysis of influenza virus mutations. A database of sequence variation generated by resequencing analysis using a resequencing array is also contemplated. Methods for monitoring viruses by variation detection using resequencing arrays are also contemplated.

For additional descriptions and methods relating to resequencing arrays see U.S. patent application Ser. Nos. 10/658,879, 60/417,190, 09/381,480, 60/409,396, 5,861,242, 6,027,880, 5,837,832, 6,723,503 and PCT Pub. No. 03/060526 each of which is incorporated herein by reference in its entirety.

The SARS virus has sequence features that are typical of the coronavirus family and sequences that distinguish this virus from other known coronaviruses. For additional information about the SARS virus see, Rota et al. Science 300:1394-1399, 2003 at sciencexpress.org, 10.1126/science.1085952 and Marra et al. Science 300:1399-1404, 2003, each of which is incorporated herein by reference.

In one embodiment an array of probes wherein each of the sequences listed in SEQ ID NOS 1-238,192 is present on the array. In one embodiment the arrays also comprise control probes such as Tag-IQ-EX probes.

In some embodiments the array may be used for rapid resequencing of SARS virus isolates from a collection of individuals. The array may be used in combination with high throughput methods, such as those disclosed in US 02/41478. Using an array as disclosed, the sequence of SARS virus isolates from at least 40 different individuals may be determined in a single day by two laboratory personnel.

U.S. Pat. Nos. 5,800,992 and 6,040,138 describe methods for making arrays of nucleic acid probes that can be used to detect the presence of a nucleic acid containing a specific nucleotide sequence. Methods of forming high-density arrays of nucleic acids, peptides and other polymer sequences with a minimal number of synthetic steps are known. The nucleic acid array can be synthesized on a solid substrate by a variety of methods, including, but not limited to, light-directed chemical coupling, and mechanically directed coupling.

In many embodiments pairs are present in perfect match and mismatch pairs, one probe in each pair being a perfect match to the target sequence and the other probe being identical to the perfect match probe except that the central base is a homo-mismatch. Mismatch probes provide a control for non-specific binding or cross-hybridization to a nucleic acid in the sample other than the target to which the probe is directed. Thus, mismatch probes indicate whether hybridization is or is not specific. For example, if the target is present, the perfect match probes should be consistently brighter than the mismatch probes because fluorescence intensity, or brightness, corresponds to binding affinity. (See e.g., U.S. Pat. No. 5,324,633, which is incorporated herein for all purposes.) Finally, the difference in intensity between the perfect match and the mismatch probe (I(PM)-I(MM)) provides a good measure of the concentration of the hybridized material. See PCT No. WO 98/11223, which is incorporated herein by reference for all purposes.

In a preferred embodiment, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids. Thus, for example, polymerase chain reaction (PCR) with labeled primers or labeled nucleotides will provide a labeled amplification product. In another embodiment, transcription amplification, as described above, using a labeled nucleotide (e.g. fluorescein-labeled UTP and/or CTP) incorporates a label into the transcribed nucleic acids. In another embodiment PCR amplification products are fragmented and labeled by terminal deoxy transferase and labeled dNTPs.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase.

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies, magnetic beads (e.g., Dynabeads™); fluorescent dyes (e.g., fluorescein, texas red, rhodamine, green fluorescent protein, and the like); radiolabels (e.g., ³H, ¹²⁵I, ³⁵S, ⁴C, or ³²P); phosphorescent labels; enzymes (e.g., horse radish peroxidase, alkaline phosphatase and others commonly used in an ELISA); and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, each of which is hereby incorporated by reference in its entirety for all purposes.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

### EXAMPLE

An array was designed to interrogate 29,774 bases of SARS sequence, the array is available as a CustomSeq™ product from Affymetrix, Inc, Santa Clara, Calif. part number 520016. The array has features that are 25×20 microns in size. The array design resequences the SARS second revision of the sequence provided by Canada's GSC (SARS_v2), as well as the variants that occur in any two of the following sequences:
1) The US CDC sequence from 4/16 (SARS_CDC)
2) The Canada GSC sequence from 4/12 (SARS_v1)
3) The HKU sequence from the Coronavirus study group from 4/16 (HKU#39849_{—}2003_{—}04_{—}16)
4) The sequence from the Chinese University of Hong Kong from 4/16 (U_HK_SARS_{—}03_{—}04_{—}16)
   From a multiple alignment of those 5 sequences, the second Canadian sequence appeared to be the best consensus sequence. It had only two variations that did not occur in at least one other sequence. The first variation is that two of the other sequences had 16 additional bases at the beginning of the sequence. Those 16 bases were pre-pended to the Canadian sequence. Then, since the last 24 bases of the sequence were a Poly-A tail, and the virus was assumed to be circular the last 12 bases were moved to the beginning of the sequence. An array was designed to resequence that full length.

Additional regions were added to resequence variants that occurred in more than one sequence. These were:
1) SARS_CDC and HKU#39849_{—}2003_{—}04_{—}16 have a single base variant at 7915
2) HKU#39849_{—}2003_{—}04_{—}16 and U_HK_SARS_{—}03_{—}04_{—}16 have a single base variant at 19049
3) All three of the non-Canadian sequences have a single base variant at 23205
4) All but SARS_v2 have a single base variant at 25283

Additionally, since HKU#39849_{—}2003_{—}04_{—}16 has a two base variant between positions 13478 and 13481 (relative to SRS_v2) a probe set was added to resequence that region. The CDC sequence had a 9 base insertion at 2835, which was not included as it has two ambiguous bases in it. The sequences are available with the following GenBank accession numbers, U_HK_SARS_{—}03_{—}04_{—}16 is now available as AY278554 (SEQ ID NO 238,193), HKU#39849_{—}2003_{—}04_{—}16 is now available as AY278491 (SEQ ID NO 238,194), SARS_v2 is now available as AY274119 (SEQ ID NO 238,195) and SARS_CDC is now available as AY278741 (SEQ ID NO 238,196). Each of the GenBank entries is incorporated herein by reference in its entirety. SARS_v1 has been retired from GenBank.

### CONCLUSION

The inventions herein provide a pool of unique nucleic acid sequences, which may be used to identify and detect variation in viral sequence such as SARS. Arrays for resequencing SARS are provided. These arrays and the resequencing data can be used for a variety of types of analyses. Databases of viral sequences generated by high throughput resequencing analysis are provided.

The above description is illustrative and not restrictive. Many variations of the invention will become apparent to those of skill in the art upon review of this disclosure. The scope of the invention should, therefore, be determined not with reference to the above description, but instead be determined with reference to the appended claims along with their full scope of equivalents.

1. An array comprising a plurality of nucleic acid probes, wherein said plurality of nucleic acid probes comprises each of the sequences listed in SEQ ID Nos. 1-238,192 wherein each different sequence is attached to the surface of the array in a different localized area.

2. A method of identifying mutations in an isolate of SARS virus comprising:
hybridizing nucleic acid derived from the isolate to the array of claim 1; and
analyzing the hybridization pattern to estimate at least 1000 bases of the sequence of the isolate.

3. A method of identifying genetic variation in a plurality of isolates of SARS virus comprising:
hybridizing nucleic acids derived from each of a plurality of isolates of the SARS virus to the array claim 1 to generate a hybridization pattern for each isolate;
analyzing the hybridization pattern for each isolate to determine a sequence of at least 1000 bases for each isolate; and
comparing the sequences to identify genetic variation.

4. An array of nucleic acid probes immobilized on a solid support, the array comprising:
(1) a first probe set comprising a plurality of probes, each probe comprising a segment of at least ten nucleotides exactly complementary to a subsequence of a SARS virus reference sequence, the segment including at least one interrogation position complementary to a corresponding nucleotide in the SARS virus reference sequence; and
(2) second, third and fourth probe sets, each probe set comprising a corresponding probe for each probe in the first probe set, the probes in the second, third and fourth probe sets being identical to the corresponding probe from the first probe set or a subsequence of at least ten nucleotides thereof that includes the interrogation position, except that the interrogation position is occupied by a different nucleotide in each of the four corresponding probes from the four probe sets.

5. The array of claim 4, wherein the probes in the first probe set have a single interrogation position, and the array further comprises a fifth probe set comprising:
a probe for each interrogation position in the first probe set, each probe in the fifth probe set being identical to a sequence comprising a corresponding probe from the first probe set or a subsequence of at least ten nucleotides thereof that includes the interrogation position, except that the interrogation position is deleted in the corresponding probe from the fifth probe set.

6. The array of claim 4, wherein the probes in the first probe set have a single interrogation position, and the array further comprises a fifth probe set comprising:
a probe for each interrogation position in the first probe set, each probe in the fifth probe set being identical to a sequence comprising the corresponding probe from the first probe set or a subsequence of at least ten nucleotides thereof that includes the interrogation position, except that an additional nucleotide is inserted adjacent to the single interrogation position in the corresponding probe from the first probe set.

7. The array of claim 4, wherein the first probe set has at least three interrogation positions respectively corresponding to each of three nucleotides in the reference sequence that are positions of known variation.

8. The array of claim 4, wherein the first probe set has at least 50 interrogation positions respectively corresponding to each of 50 nucleotides in the reference sequence that are positions of known variation.

9. The array of claim 4, wherein the array has between 10,000 and 1,000,000 probes.

10. The array of claim 4, wherein the array has between 1,000,000 and 2,600,000 probes.

11. The array of claim 4, wherein the array has between 2,600,000 probes and 20,000,000 probes.

12. The array of claim 4, wherein the segment in each probe of the first probe set that is exactly complementary to the subsequence of the reference sequence is 9 to 35 nucleotides.

13. The array of claim 4 wherein the array interrogates at least 1000 contiguous bases of a SARS virus for variation.

14. The array of claim 4 further comprising:
(1) a fifth probe set comprising a plurality of probes, each probe comprising a segment of at least ten nucleotides exactly complementary to a subsequence of a second reference sequence, the segment including at least one interrogation position complementary to a corresponding nucleotide in the second reference sequence; and
(2) sixth, seventh and eighth probe sets, wherein the sixth, seventh and eigth probe sets each comprise a corresponding probe for each probe in the fifth probe set, the probes in the sixth, seventh and eighth probe sets being identical to the corresponding probe from the fifth probe set, or a subsequence of at least ten nucleotides thereof that includes the interrogation position, except that the interrogation position is occupied by a different nucleotide in each of the four corresponding probes from the fifth, sixth, seventh and eighth probe sets.

15. The array of claim 14 wherein the second reference sequence is an influenza viral sequence.

16. A method of monitoring genetic variation of SARS virus in a population of individuals comprising:
acquiring viral isolates from a plurality of individuals suspected of being infected with the virus;
estimating the sequence of at least one virus in each viral isolate by hybridizing nucleic acid derived from the viral isolate to the array of claim 4; and
comparing the estimated sequences to identify the presence or absence of variation between individual isolates.

17. The method of claim 16 wherein one or more steps of the method are performed in a high throughput assay.

18. A method of generating a database of viral sequences comprising:
isolating viral samples from a plurality of sources;
hybridizing nucleic acid derived from each of the viral samples to the array of claim 4 to generate a hybridization pattern for each viral sample;
determining the sequence of each viral sample from the hybridization pattern; and
combining the sequence of each viral sample from each source in the plurality of sources into a database of viral sequences.

19. A method of controlling an outbreak of SARS comprising:
estimating the sequence of isolates of SARS virus from a plurality of affected individuals using the array of claim 4;
comparing the sequences to a database of sequences of viral strains that are associated with high rates of transmission;
identifying one or more individuals carrying an isolate of the virus that is known to be associated with high rates of transmission; and
minimizing the contact between said one or more individuals and unaffected individuals.

20. A method of limiting the mortality or morbidity resulting from an outbreak of SARS comprising:
estimating the sequence of isolated of SARS virus from affected individuals using the array of claim 4;
comparing the sequences to a database of SARS virus sequences that are associated with high rates or mortality or morbidity;
identifying one or more individuals carrying a SARS virus that is associated with high rates of mortality or morbidity; and
minimizing the contact between said one or more individuals and unaffected individuals.

21. A method of monitoring an outbreak of a disease caused by a virus of interest comprising:
isolating a nucleic acid sample from each of a plurality of individuals suspected of being infected with the virus of interest;
amplifying viral nucleic acid from the virus of interest in each of the nucleic acid samples;
hybridizing the amplified nucleic acids to a resequencing array comprising probes to a reference sequence of the virus of interest;
estimating at least part of the sequence of the virus of interest in each of the samples; and
comparing the sequences to determine the variation between individual isolates.

22. The method of claim 21 wherein the virus of interest is a SARS virus.

23. The method of claim 21 wherein the virus of interest is an influenza virus.

24. The method of claim 21 wherein one or more steps of the method are performed in a high throughput assay.

25. An array of nucleic acid probes immobilized on a solid support, the array comprising at least two sets of probes,
(1) a first probe set comprising a plurality of probes wherein each probe comprises a segment of at least ten nucleotides that is perfectly complementary to a subsequence of a first reference sequence, the subsequence including at least one interrogation position complementary to a corresponding nucleotide in the first reference sequence,
(2) a second probe set comprising a plurality of probes wherein each probe comprises a subsequence of at least ten nucleotides that is perfectly complementary to a subsequence of a second reference sequence, the subsequence including at least one interrogation position complementary to a corresponding nucleotide in the second reference sequence, wherein the first reference sequence is a first isolate of a SARS virus and the second reference sequence is a second isolate of a SARS virus.

26. The array of claim 25, wherein the first reference sequence is from a super spreader isolate of SARS.

27. A method of comparing a target nucleic acid with a reference sequence, the method comprising:
(a) hybridizing a sample comprising the target nucleic acid to the array of claim 25;
(b) comparing the hybridization pattern of the two corresponding probes from the first and second probe sets;
(c) assigning a nucleotide in the target sequence as the complement of the interrogation position of the probe having the greater hybridization; and
(d) repeating (b) and (c) by comparing the hybridization pattern of a further two corresponding probes from the first and second probe sets until each nucleotide of interest in the target sequence has been assigned.

28. A method of comparing a target nucleic acid with a reference sequence comprising a predetermined sequence of nucleotides, the method comprising:
(a) hybridizing a sample comprising the target nucleic acid to an array of nucleic acid probes immobilized on a solid support, the array comprising at least four sets of probes,
(1) a first probe set comprising a plurality of probes, each probe comprising a segment of at least nine nucleotides exactly complementary to a subsequence of a reference sequence, the segment including at least one interrogation position complementary to a corresponding nucleotide in the reference sequence,
(2) second, third and fourth probe sets, each set comprising a probe for each interrogation position in the first probe set, each probe comprising a corresponding probe for each probe in the first probe set, the probes in the second, third and fourth probe sets being identical to a sequence comprising the corresponding probe from the first probe set or a subsequence of at least nine nucleotides thereof that includes the interrogation position, except that the interrogation position is occupied by a different nucleotide in each of the four corresponding probes from the four probe sets;
provided the array does not consist of a complete set of probes of a given length, wherein a complete set is all permutations of nucleotides A, C, G and T/U;
wherein the reference sequence is a SARS virus
(b) comparing the relative specific binding of four corresponding probes from the first, second, third and fourth probe sets;
(c) assigning a nucleotide in the target sequence as the complement of the interrogation position of the probe having the greatest specific binding;
(d) repeating (b) and (c) by comparing the relative specific binding of a further four corresponding probes from the first, second, third and fourth probe sets until each nucleotide of interest in the target sequence has been assigned.

29. The method of claim 28 wherein one or more steps of the method are performed in a high throughput assay.
